(19) United States
(12) Patent Application Publication
(10) Pub. No.: US 2025/0332005 A1
(43) Pub. Date: Oct. 30, 2025

(54) PUMP MECHANISM

(71) Applicant: Ossur Iceland ehf, Reykjavik (IS)

(72) Inventors: Snorri Rafn THEODORSSON, Reykjavik (IS); Marco STEINBERG, Reykjavik (IS); Victoria Pamela Anne CLARK, Reykjavik (IS); Christophe Guy LECOMTE, Reykjavik (IS)

(21) Appl. No.: 19/192,605

(22) Filed: Apr. 29, 2025

Related U.S. Application Data
(60) Provisional application No. 63/639,843, filed on Apr. 29, 2024.

(51) Int. Cl. A61F 2/66; A61F 2/50; A61F 2/80

(52) U.S. Cl. CPC A61F 2/66; A61F 2/80; A61F 2002/5081; A61F 2002/6671; A61F 2002/6678; A61F 2002/805

(57) ABSTRACT

The present disclosure pertains to prosthetic systems that encompass a prosthetic foot and a pump mechanism devised to eliminate fluid between a prosthetic socket and a residual limb or liner, thereby sustaining a hypobaric pressure chamber within a cavity of the prosthetic socket. The pump mechanism may incorporate a fluid chamber established between a membrane and the housing of the pump mechanism. Forces resulting from the user's gait during the compression and expansion of the prosthetic foot can be transmitted to an L-brace, which is configured to actuate the membrane of the pump mechanism, facilitating the influx and efflux of fluid into and from the fluid chamber.

### TECHNICAL FIELD

The present disclosure pertains to prosthetic devices, specifically focusing on systems and pump mechanisms that enhance vacuum levels within vacuum-assisted suspension systems.

### BACKGROUND

A notable challenge in the development of prosthetic devices is the effective attachment of these devices to a user's residual limb. In the case of prosthetic legs, achieving a secure attachment to the residual limb without applying excessive or uneven pressure is often problematic. While a secure connection is essential for optimal walking functionality, an improper fit can result in sores, swelling, and pain for the user.

A prevalent method for addressing this challenge involves the application of a negative pressure vacuum within the space between the limb (or a liner worn on the limb) and a socket that is connected to the prosthetic limb. Two conventional approaches to creating such a vacuum are the use of mechanical pumps and electronic pumps.

Mechanical pumps generally operate as in-line systems, utilizing the user's movements to generate negative pressure within the socket. For example, the force produced by the user's foot contacting the ground during walking can create a vacuum that secures the prosthesis to the residual limb. However, these pumps necessitate complete compression to expel air before being able to decompress and generate a vacuum. Due to the variability in the impact and displacement of the pump among different users, the resulting vacuum- and consequently the attachment between the residual limb and the socket—can be inconsistent or inadequate, potentially leading to discomfort, dissatisfaction, and even injury.

Moreover, conventional in-line systems are typically situated between the socket and the prosthetic limb. This positioning can negatively impact the height of the prosthesis during walking, diminishing user comfort, and restricting the available height of the prosthetic foot, which limits configuration options. Additionally, it is worth noting that mechanical pump mechanisms are sometimes installed directly on the prosthetic foot.foot.

However, current configurations are generally oriented in such a way as to make the connection of vacuum tubing to the pump mechanism difficult (while also increasing the likelihood that the vacuum tubing will catch and snag on environmental obstacles and the flexible portions of the prosthetic foot). Additionally, pump mechanisms have been placed on the prosthetic foot at locations that expose the pump mechanism (e.g., on the dorsal portion of the prosthetic foot or at the heel), such that damage to the pump mechanism is increased.

Accordingly, a pump mechanism positioned on the prosthetic foot at a favorable location for protecting it, easily removing it in case of damage, and keeping it in a protected location is needed. A prosthetic device that provides a secure vacuum without losing suction and confidence to the user over a period of use is also needed. It is also desirable for prosthetic devices to draw a vacuum while being lightweight, streamlined, and versatile.

### SUMMARY

A prosthetic system is disclosed herein designed to maintain a hypobaric pressure chamber between the prosthetic socket and a residual limb. The prosthetic system comprises a pump mechanism operatively connectable to a prosthetic foot equipped with an adaptor. The pump mechanism includes a housing featuring an interior cavity, a one-way valve assembly connected to the housing and in fluid communication with the interior cavity, a membrane at least partially located within the interior cavity, a connector affixed to the membrane and extending outside the interior cavity, and an L-brace attached to both the housing and the prosthetic foot. The housing may be positioned at or near the proximal end of the prosthetic foot beneath the adaptor, while the interior cavity may be arranged at or near the distal end of the housing.

The L-brace may be designed to cooperate with the prosthetic foot to actuate the membrane. Specifically, relative movement of the L-brace against the prosthetic foot may exert a pulling force via the connector on the membrane to draw fluid from a cavity of a prosthetic socket through the one-way valve assembly. The L-brace may include a first curve characterized by a radius of curvature that opens towards the prosthetic foot, with the radius of curvature of the first curve being opposite to that of the prosthetic foot. A proximal end of the L-brace may be affixed to a posterior section of the housing.

The interior cavity may feature an undercut circumferential groove positioned between an open end and a top of the interior cavity, enabling an outer radial edge of the membrane to reside within the undercut circumferential groove, thus forming a seal between the membrane and the housing. The membrane may be movable between a contracted configuration, in which the volume of a fluid chamber defined between the membrane and the top of the interior cavity is zero or nearly zero, and an expanded configuration, wherein the fluid chamber volume increases.

In another embodiment, the prosthetic system may consist of a pump mechanism including a housing possessing an interior cavity, a one-way valve assembly linked to the housing and in fluid communication with the interior cavity, a membrane at least partially contained within the interior cavity, and a connector affixed to the membrane and protruding out of the interior cavity. Additionally, a C-blade pivotably connected to the housing may be configured for attachment to the proximal end of the prosthetic foot. The C-blade may exhibit a curvature aligned in the same direction as that of the prosthetic foot. The C-blade and housing may function collaboratively with the prosthetic foot to actuate the pump mechanism.

The posterior section of the housing may engage with the distal end of the C-blade when the pump mechanism is in a contracted configuration. The interior cavity may be situated on the posterior section of the housing and is adaptable to receive a membrane attached to the distal end of the C-blade. A bump component extending from the posterior section of the housing may slidably contact the dorsal portion of the prosthetic foot. During the expansion of the pump mechanism, the C-blade may pivot forward as a result of the user's gait, causing the distal end of the C-blade to shift downward. As the distal end descends, the bump component may push against the dorsal section of the prosthetic foot, elevating the housing and thereby separating the top surface of the membrane from the top of the interior cavity to augment the volume of the fluid chamber situated between them, thus actuating the pump mechanism.

The prosthetic system may comprise a prosthetic foot with a dual-blade configuration. Furthermore, the foot may include a heel member extending along the distal portion of the foot to a point posterior to the exterior surface of the dual blades. The system may also incorporate a heel component located between the heel member and the dual blades of the foot.

These and other features, aspects, and advantages of the present disclosure will be better comprehended in the ensuing description, appended claims, and accompanying drawings.

### BRIEF DESCRIPTION OF THE DRAWINGS

The drawing figures are not necessarily drawn to seale but instead are drawn to provide a better understanding of the components thereof and are not intended to be limiting in scope but to provide exemplary illustrations. The figures illustrate exemplary configurations of prosthetic systems and pump mechanisms, and in no way limit the structures or configurations according to the present disclosure.

FIG. 1 shows an exemplary prosthetic device.

FIG. 2 shows a side view of an exemplary prosthetic system, having a prosthetic foot, a prosthetic connector, and a pump mechanism.

FIG. 3 shows a perspective view of an exemplary pump system.

FIG. 4 shows a perspective view of an L-brace.

FIG. 5 shows an exploded bottom perspective view of the pump housing, membrane, and connector.

FIG. 6 shows a transparent view of the pump housing.

FIG. 7 shows an exploded perspective view of the pump housing.

FIG. 8A shows a sectional view of a pump mechanism in a contracted configuration.

FIG. 8B shows a sectional view of a pump mechanism in an expanded configuration.

FIG. 9A shows a sectional view of a pump mechanism in a contracted configuration.

FIG. 9B shows a sectional view of a pump mechanism in an expanded configuration.

FIG. 10 shows a side view of an exemplary prosthetic system, having a prosthetic foot, a prosthetic connector, and a pump mechanism.

FIG. 11 shows a perspective view of an exemplary pump mechanism.

FIG. 12 shows an exploded bottom perspective view of the pump housing, membrane, and connector.

FIG. 13A shows a sectional view of a pump mechanism in a contracted configuration.

FIG. 13B shows a sectional view of a pump mechanism in an expanded configuration.

### DEFINITIONS

The term “anterior” refers to portions of the prosthetic system disposed on a front-facing side of the system.

The term “posterior” refers to portions of the prosthetic system disposed to the rear of the system.

The term “proximal” refers to portions of the prosthetic system located closer to an adaptor of the prosthetic foot or alternatively refers to portions of the prosthetic system positioned closer to the heart when worn by a user.

The term “distal” refers to portions of the prosthetic system located closer to a toe end of the prosthetic foot or to portions of the prosthetic system positioned further from the heart when worn by a user.

### DETAILED DESCRIPTION OF VARIOUS EMBODIMENTS

It will be understood that unless a term is expressly defined in this disclosure to possess a described meaning, there is no intent to limit the meaning of such term, either expressly or indirectly, beyond its plain or ordinary meaning.

Any element in a claim that does not explicitly state “means for” performing a specified function, or “step for” performing a specific function, is not to be interpreted as a “means” or “step” clause as specified in 35 U.S.C. § 112.

Throughout the detailed description, features labeled with a particular reference number may exhibit characteristics similar to other features bearing a similar reference number.

The embodiments of a prosthetic device will be described, which form part of a vacuum system. A vacuum pump mechanism with a fluid connection to a socket assists in creating a vacuum between the socket and a residual limb and/or liner by pumping fluid out of the socket. The fluid is pumped out of the socket when the user puts his weight on the prosthetic foot, such as upon heel strike, mid-stance, and toe-off. The user's weight on the prosthetic foot can cause the pump mechanism to increase the volume of the fluid chamber in the pump. The increased volume of the pump mechanism draws fluid from the vacuum space between the residual limb and the prosthetic limb socket. In this way, the pump mechanism reduces the air pressure within the vacuum space, creating a vacuum effect.

After the weight is removed and/or shifted on the prosthetic foot, the volume of the fluid chamber in the pump mechanism is automatically decreased. In some embodiments, a user's weight on the prosthetic foot can cause the pump mechanism to expel fluid from the fluid chamber of the pump mechanism. The connection between the vacuum space and the pump may include a one-way valve assembly, so all the air within the pump volume is expelled from an outlet to another space or the atmosphere. The outlet features a one-way valve assembly, ensuring that the vacuum space is the only air source.

The vacuum suspension system of the present disclosure produces a vacuum effect in a prosthetic socket that is advantageous over prior art devices requiring compression of the pump to expel air before the pump can be decompressed to draw in air. The present disclosure also achieves smaller fluctuations in air pressure compared to prior art systems, so the difference between the highest and lowest pressure in the vacuum space of the socket is minimized.

The efficiency of the pump mechanism is determined, at least in part, by how effectively the volume of the fluid chamber is reduced. Since the pump mechanism begins at and returns to its original state of zero or near-zero volume at the beginning or end of each cycle in some embodiments, the volume of the fluid chamber depends on the force applied to the pump, rather than a full compression and recompression cycle as in the prior art. Moreover, all fluid drawn into the pump mechanism is expelled afterward, fully utilizing the volume of the fluid chamber.

The vacuum suspension system also reduces the volume fluctuations of the residual limb. It allows for increased proprioception and diminished pistoning, as there is a better attachment between the socket and the residual limb. Producing hypobaric pressure below a certain level in the socket may also be beneficial. This may be achieved using a sealing membrane or seal component between the residual limb and the socket instead of the conventional method of employing a sleeve to create an airtight connection between the residual limb and the proximal end of the socket. The sealing membrane may be on a prosthetic liner as described in U.S. Pat. Nos. 8,034,120; 8,097,043; and 9,066,821, each incorporated by reference and belonging to the assignee of this disclosure.

The benefit of using a liner with a seal or seal component is that it reduces the volume of air drawn out of the socket, allowing for better suspension in a shorter period. Using a silicone liner with an integrated seal also provides the added benefit that the hypobaric region is not directly applied to the skin.

The vacuum pump mechanisms in the embodiments of the prosthetic device are generally described as pump mechanisms and may include any suitable type of pump mechanism. A piston-type pump may be used instead of a membrane-type pump in these embodiments. A bladder-type pump may also be used in place of a membrane-type pump, and a skilled person would understand that the described pump mechanisms may also work with a bladder-type pump and vice versa. The pump mechanism in the embodiments of the prosthetic device is generally located at or near the proximal portion of the prosthetic foot, such as on the bottom surface of the elongated plate foot members. The pump mechanisms will also generally be positioned such that they actuate in a proximal-distal direction. For example, the volume of a fluid chamber may increase as a portion of the pump (e.g., a membrane, bladder portion, or piston) translates in a generally distal direction.

A bladder-type pump has an interior fluid chamber surrounded by an airtight material. When the interior chamber expands, the opposing walls move away from each other by extending at least one side wall of the pump. The side walls of the bladder-type pump may have an accordion-like shape or be formed of a polymeric material, which allows for an increase in the distance between the opposing walls.

A membrane-type pump has at least one wall made of flexible material and a second opposing wall, which may be rigid or flexible. The edges of the two walls are attached such that when a force is applied to the pump to expand the interior fluid chamber, the force deforms at least the flexible wall, and the flexible wall arcs outward to form the interior fluid chamber. To allow for deformation, the flexible wall may be made of a polymeric material, including elastomeric materials such as rubber or plastic.

The bladder-type pump and membrane-type pump may be arranged so that when no force is applied to the pump, or no weight is placed on the prosthetic device, the volume of the interior fluid chamber is zero or near zero. In other embodiments, the pumps may be arranged so that when no force is applied, the volume of the interior fluid chamber is greater than zero. The pumps described and shown have a cylindrical shape. A skilled person would understand that the pumps may have a variety of shapes, such as diamond, rectangular, or triangular shapes.

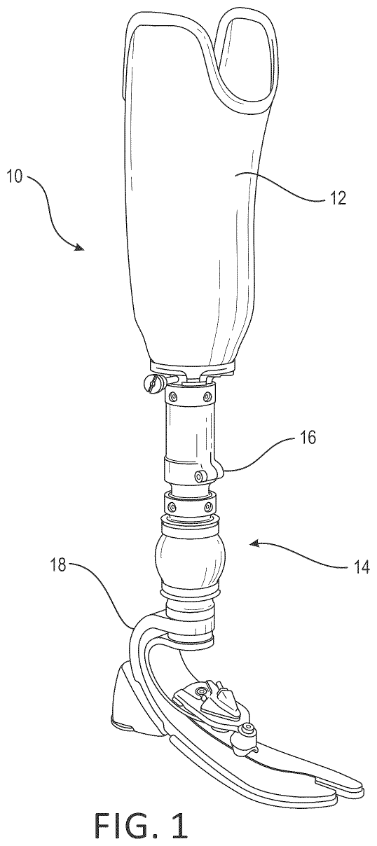

FIG. 1 shows an exemplary prosthetic device 10 that may include prosthetic systems (such as prosthetic feet and pump mechanisms) relating to this disclosure. The prosthetic device may comprise a prosthetic socket 12 and a prosthetic limb 14 including a pylon 16 and a prosthetic foot 18. The prosthetic socket 12 is configured to receive the residual limb of a user. A hypobaric pressure chamber formed between the socket 12 and the residual limb and/or liner may beneficially secure the socket 12 to the residual limb, and may beneficially increase comfort, improve rotation transmitted from the residual limb to the prosthetic limb 14, and enhance proprioception of the user. A liner may be donned over the residual limb of the user, which may interface between the residual limb and the prosthetic socket 12.

The formation of the hypobaric pressure chamber may benefit from a seal between the residual limb and the socket 12. This seal can be created with the aid of a seal ring or seal component positioned between the residual limb and the socket 12. The hypobaric pressure chamber is established as the user inserts the residual limb into the socket 12, with air between the socket 12 and the residual limb being pushed out through a one-way valve. However, movement between the residual limb and the socket 12 during the use of the prosthetic device may cause an imperfect seal, allowing air to enter between the residual limb and the socket 12 and reducing the strength of the hypobaric pressure chamber.

Devices in the prior art have aimed to address this issue using pump mechanisms, including both electronic and mechanical types. Mechanical pump mechanisms offer advantages over electronic ones as they do not require batteries and can utilize forces generated by the user's gait to operate the pump. Typically, these pump mechanisms are placed in-line between a prosthetic socket and a prosthetic foot of a prosthetic device, such as between the prosthetic socket and a pylon. For instance, the force applied to the prosthetic foot is transmitted through the pylon to the pump mechanism, compressing it and creating a vacuum that draws fluid from the socket to maintain the hypobaric pressure chamber. However, these in-line pump mechanisms reduce the space between the prosthetic socket and the prosthetic foot, limiting the height available to the pylon and prosthetic foot, and thereby restricting the configurations and/or dampening characteristics of the prosthetic device. Furthermore, compression of the pump mechanism tends to alter the height of the prosthetic device, which may cause discomfort and reduced proprioception for the user.

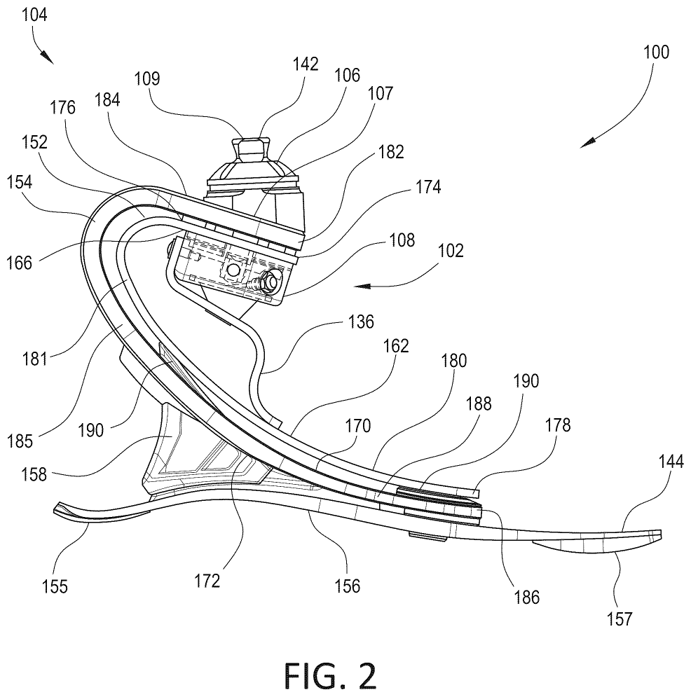

FIG. 2 illustrates an embodiment of a prosthetic system 100 that includes a pump mechanism 102 and a prosthetic foot 104 with dual foot blades featuring elongated plates. The prosthetic foot 104 contains an inner foot member 152 that extends from a first end portion 176, terminating at a first or proximal end 174, to a second end portion 180, which terminates at a second or distal end 178. The first end portion 176 can be generally horizontally oriented or positioned at an oblique angle to the horizontal, while the second end portion can also be generally horizontally oriented.

The inner foot member 152 can have an intermediate portion 181 that extends between the first end portion 176 and the second end portion 180. This intermediate portion 181 can feature a flexible configuration and define a curvature. The intermediate portion 181 is typically forwardly facing concave, giving the inner foot member 152 a generally C-shaped design. The intermediate portion 181 and/or the first end portion 176 can be located in a position similar to that of a natural human ankle.

The prosthetic foot 104 may include an outer foot member 154 that generally encases the inner foot member 152, positioned posterior to the inner foot member 152. The outer foot member 154 extends from a first end portion 184, which terminates at a first or proximal end 182, to a second end portion 188, which ends at a second or distal end 186. The first end portion 184 can be generally horizontally oriented or set at an oblique angle to the horizontal, with the second end portion 188 also being generally horizontally oriented. The outer foot member 154 can define an intermediate portion 185 between the first end portion 184 and the second end portion 188. This intermediate portion 185 can also have a flexible configuration and define a curvature, with the intermediate portion 185 typically forwardly facing concave, resulting in the outer foot member 154 having a generally C-shaped contour.

The prosthetic foot 104 can have a heel member 156 that extends rearward from a first or anterior end 157 to a free second or posterior end 155 and is positioned below at least a portion of the outer foot member 154. In some embodiments, the heel member 156 may extend along at least a portion or to the entirety of the second portion of the outer foot member 154. The heel member 156 can have a curvilinear profile along its length.

In the illustrated embodiment, the inner foot member 152 and the outer foot member 154 extend parallel to each other and generally have the same shape. Intermediate portions 181 and 185 of the inner and outer foot members 152 and 154 can have predetermined lengths to provide the prosthetic foot 104 with the desired flexibility. The curvature of the outer foot member 154 can follow or be generally concentric with that of the inner foot member 152. The second or distal end 186 of the outer foot member 154 may extend beyond the second or distal end 178 of the inner foot member 152. The outer foot member 154 may be coupled to the heel member 156 at the second end portion 188 of the outer foot member 154. The inner foot member 152 may rest upon spacer pads 190 placed between the inner foot member 152 and an interior surface 170 of the outer foot member 154, allowing the inner foot member 152 to slide relative to the outer foot member 154.

An adaptor 106 can be coupled to the inner foot member 152 and the outer foot member 154. The inner and outer foot members 152 and 154 may be attached to a distal end 107 of the adaptor 106 through one or more fasteners. In some embodiments, the adaptor 106 can include a cavity sized and shaped to receive an attachment portion of the prosthetic foot 104, such as the posterior or first ends of the outer and inner foot members 154 and 152. The adaptor 106 can include a cavity located on its distal end 107. The adaptor 106 may comprise a prosthetic connection 109, such as a male pyramid connection, for connecting the prosthetic foot 104 to a prosthetic device.

In use, the prosthetic foot 104 can expand and compress through flexion of the inner and outer foot members 152 and 154. The prosthetic foot 104 is in expansion when the first and second end portions 176 and 180 of the inner foot member 152 and the first and second end portions 184 and 188 of the outer foot member 154 are moved or flexed apart from a resting position, increasing the distance between the first 176, 184 and second 180, 188 end portions of the inner and outer foot members 152 and 154. The prosthetic foot 104 is in compression when the first 176, 184 and second 180, 188 end portions of the inner and outer foot members 152 and 154 are moved or flexed toward one another from the resting position, decreasing the distance between the first 176, 184 and second 180, 188 end portions of the inner and outer foot members 152 and 154.

To better understand the operation of the prosthetic foot 104, a basic discussion of the gait cycle is necessary. The gait cycle defines the movement of the leg between successive heel contacts of the same foot. It has two phases: stance and swing. Of particular interest is the stance phase, which generally includes the stages of heel-strike or initial contact, mid-stance, and toc-off.

It is during the stance phase that the mechanics of the prosthetic foot 104 come into play. Upon heel strike, the prosthetic foot 104 is in expansion, providing cushioning to the user. During mid-stance, when the weight of the user is transmitted through the prosthetic foot 104 to a supporting surface, the prosthetic foot 104 transitions from expansion into compression. The prosthetic foot 104 remains in compression through toe-off until the weight of the user is removed, at which point the prosthetic foot 104 returns to its resting position.

The pump mechanism 102 can be coupled to the prosthetic foot 104 at any suitable location, but it is shown coupled to the adaptor 106 located at the first or proximal ends 174 and 182 of the inner and outer foot members 152 and 154. The pump mechanism 102 can be made primarily from carbon fiber and an elastomeric compound (e.g., a membrane 126), providing durable yet lightweight components. In contrast to prior art pump mechanisms, which are made of heavy metal construction, this design significantly reduces the weight burden on the user while walking.

The pump mechanism 102 is situated between the adaptor 106 and an L-brace 136 that engages the inner foot member 152. As described in more detail below, the relative movement of the L-brace 136 can shift the pump mechanism 102 between a compressed configuration and an expanded configuration.

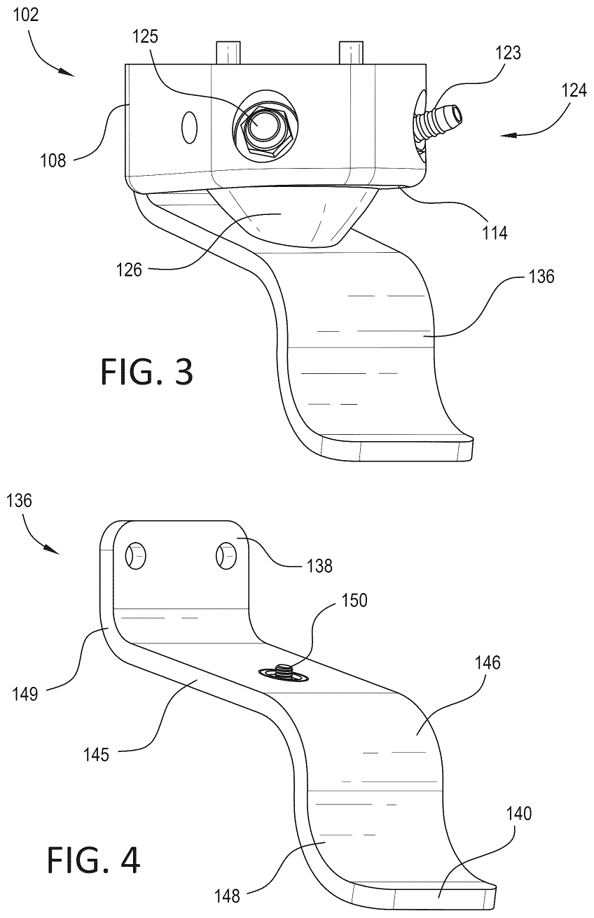

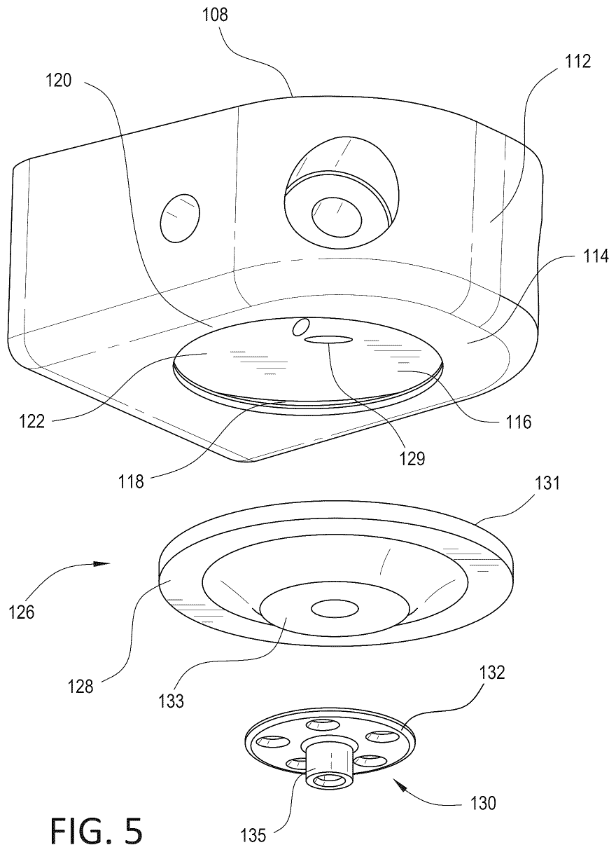

FIGS. 3-6 illustrate an exemplary pump mechanism 102. The pump mechanism 102 includes a housing 108 with an interior cavity 116 that contains a valve assembly 124, a membrane 126 (shown in FIG. 5), and a connector 130 (shown in FIG. 5). The housing 108 may be attached to the bottom surface of the prosthetic foot 104, such as the bottom surface 166 of the inner foot member 152, or at a location on the distal end 107 of the adaptor 106. This placement of the housing 108 beneficially protects it during walking motions, reducing the likelihood of detrimental contact with objects at or near ground level or from impacts from above. The interior cavity 116 may be formed at a distal end 114 of the housing 108. A fluid chamber 134 may be created between the interior cavity 116 and the membrane 126 at the distal end 114 of the housing 108, positioned above the membrane 126. The valve assembly 124 can include a one-way valve, also known as a check valve. A preferred type of one-way valve is the duckbill valve; however, it should be noted that other types of one-way valves are also possible.

The valve assembly 124 may be disposed at an anterior portion 112 of the housing 108. An inlet 123 of the valve assembly 124 can be in fluid communication with the cavity of a prosthetic socket via a tube (not shown) and is arranged to only allow fluid to enter the pump mechanism 102. When the volume of the fluid chamber 134 increases, fluid (e.g., air) can be drawn out from the socket via the valve assembly 124. An outlet 125 of the valve assembly 124 may also comprise a one-way valve, such that the outlet 125 is arranged to only allow fluid to be expelled out of the pump mechanism 102 preferably to atmosphere.

The housing 108 can be coupled to the adaptor 106 via at least one fastener. An upper surface of the housing 108 can generally complement the lower surface of the first or proximal end 174 of the inner foot member 152. It should be appreciated that the pump mechanism 102 can be a separate add-on module to the prosthetic foot 104. For instance, the pump mechanism 102 can be removably coupled to the adaptor 106 and/or inner and outer foot members 152 and 154 via a fastener and to the L-brace 136. Because the pump mechanism 102 is not integrated into the prosthetic foot 104 failure of the pump mechanism 102 advantageously would not affect the performance of the prosthetic foot 104. The housing 108 can have a rigid configuration.

FIG. 4 shows the L-brace 136 which facilitates actuation of the pump mechanism 102. The L-brace 136 may include a first curve 146 that defines a curvature in a direction opposite to the direction of curvature of the inner foot member 152. The L-brace 136 may also comprise a second curve 148 distal of the first curve 146 defining a curvature in a direction opposite of the first curve 146. The first and second curves 146 and 148 beneficially facilitate expansion and compression of the L-brace 136. The distal end 140 of the L-brace 136 may interface with the inner foot member 152 to aid in actuating the pump mechanism 102. The distal end 140 of the L-brace 136 may slidably contact the inner foot member 152. In some embodiments, the distal end 140 of the L-brace 136 is attached to the inner foot member 152, such as through a fastener or an adhesive.

The proximal end 138 of the L-brace 136 is connected to the housing 108 or prosthetic foot 104 at a point posterior to the membrane 126, such as a posterior portion 110 of the housing 108. A third bend 149 may enable the proximal end 138 of the L-brace 136 to connect to the posterior portion 110 of the housing 108 and allow the L-brace 136 to extend across the distal end 114 of the housing 108. The L-brace 136 may comprise an actuation component 150 (such as a screw or other proximally protruding surface) protruding from an intermediate portion of the L-brace 136 configured to engage a connector 130 attached to the membrane 126. When the prosthetic foot 104 is in the original configuration (e.g., resting position during swing phase) the L-brace 136 may be in a compressed state, such that the L-brace exerts a force against a dorsal portion 162 of the inner foot member 152.

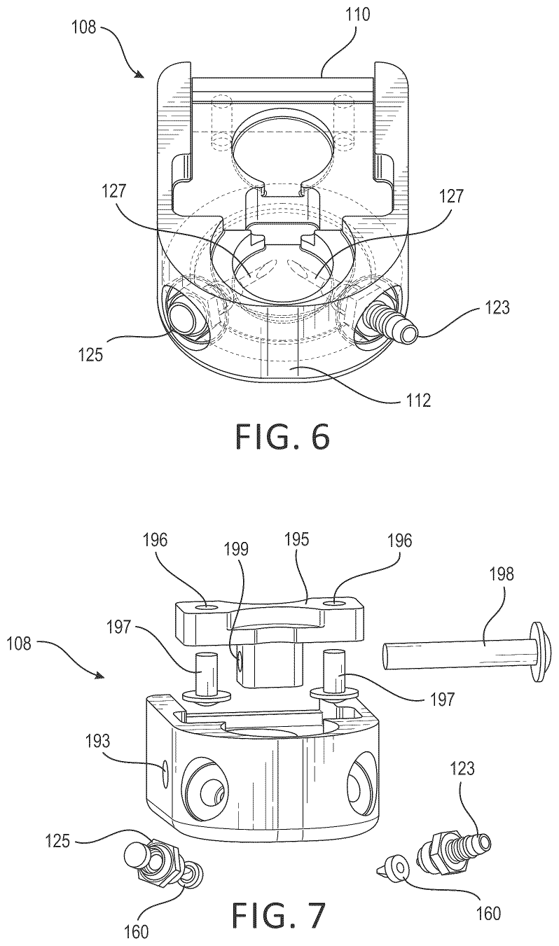

Referring to FIG. 5, the bottom surface of the housing 108 defines an interior cavity 116 that is provided with an undercut circumferential groove 118 between an open end 120 of the interior cavity 116 and a closed top 122 of the interior cavity 116. An outer radial edge portion 128 of the membrane 126 can be situated in the circumferential groove 118, forming a seal between the membrane 126 and the housing 108. Optionally, an adhesive can be applied between the housing 108 and the outer radial edge portion 128 of the membrane 126, increasing the sealing effect. The top 122 of the interior cavity 116 has two openings 129 (shown in FIG. 5) which extend into the housing 108 to form internal passageways 127 (shown in FIG. 6) providing fluid communication between a fluid chamber 134 defined below and the one-way valve assembly 124. The housing 108 may be formed of metal such as stainless steel or aluminum, carbon fiber, glass fiber, or plastic or any other material which would provide sufficient strength to resist deformation when pulled away from the membrane 126. The housing 108 may also be formed from additive manufacturing, injection molding, or extrusion processes.

A connector 130 may be partially disposed within the membrane 126 and protrude from the membrane 126 and the interior cavity 116 of the housing 108. The connector 130 can be an insert with a radial flange 132 to secure the connector 130 to the membrane 126. The connector may also include a shaft 135 protruding from the bottom surface 133 of the membrane 126 configured to attach to the actuation component 150 protruding from an intermediate portion of the L-brace 136. The connector 130 may also comprise an additional flange (not shown) disposed at the distal end of the connector 130 to facilitate connection to the actuation component 150. In some embodiments, the insert may be of a two-piece construction such that the additional flange can be threadedly removed from the radial flange 132 embedded in the membrane 126. The connector 130 may be formed of metal, plastic, or any suitable other material. In other embodiments, the additional flange of the insert may extend substantially into the membrane 126 or may be formed of a material that is part of the membrane 126 (e.g., a flexible metal member).

FIG. 7 shows an exploded view of the pump housing 108. The housing 108 may comprise an insert 195 configured to facilitate easy attachment of the housing 108 to the prosthetic foot 104. The insert 195 may comprise openings 196 through which a fastener (such as bolts 197) may be threaded and secured to the proximal end 142 of the prosthetic foot 104. The insert 195 may be secured to the housing 108 via bolt 198, or other fastener, which may pass through openings 193 and 199. FIG. 7 also illustrates the one-way valves 160 in fluid communication with the inlet 123 and outlet 125 of the housing 108.

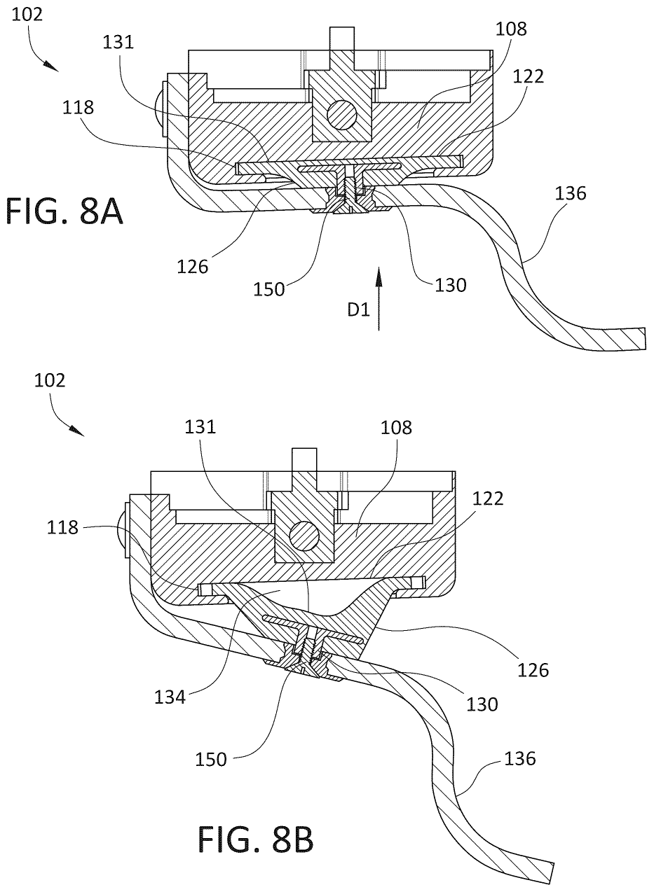

FIGS. 8A-8B illustrate the actuation of the pump mechanism 102. The pump mechanism 102 is movable between a contracted configuration (shown in FIG. 8A) in which the volume of a fluid chamber 134 defined between the top surface 131 of the membrane 126 and the top 122 of the interior cavity 116 is zero or near-zero, and an expanded configuration (shown in FIG. 8B) in which the volume of the fluid chamber 134 is increased. The contracted configuration may be the original configuration of the prosthetic system 100 (i.e., the configuration of the prosthetic system 10 when no load is placed on the prosthetic foot 104, such as during the swing phase). In other embodiments, the original configuration may be an expanded configuration.

As seen, the top 122 of the interior cavity 116 substantially complements the top surface 131 of the membrane 126 such that when a force is exerted on the pump mechanism 102 it is in the contracted configuration. The top 122 of the interior cavity 116 and the top surface 131 of the membrane 126 can be generally flat. The pump mechanism 102 may then move to the expanded configuration when no external force is exerted on the pump mechanism 102.

The pump mechanism 102 may be actuated during the stages of the stance phase and/or swing phase. In the swing phase, the pump mechanism 102 may be in the expanded configuration such that the volume of the fluid chamber 134 is greater than zero. On heel strike or initial contact, the heel member 156 first contacts the support surface (e.g., the ground). During this stage, the prosthetic foot 104 moves into expansion, with a distance between the first or proximal end 174 and the second or distal end 178 of the inner foot member 152 increasing, the curvature of the inner foot member 152 widening. The outer foot member 154 may undergo an expansion like the inner foot member 152. Expansion of the prosthetic foot 104 enables a corresponding expansion of the L-brace, the L-brace 136 extending to a larger length than during the original configuration.

As the heel strike transitions to mid-stance and toc-off, the prosthetic foot is compressed with the distance between the first or proximal and second or distal ends 174 and 178 of the inner foot member 152 decreasing to a shorter distance when compared with the original configuration to compress the inner foot member 152. Forces from the compression of the inner foot member 152 are transmitted to the L-brace 136 such that the L-brace 136 is also compressed. During compression of the L-brace 136 the first and second curves 146 and 148 may bend to accommodate the shortening distance between the dorsal portion of the inner foot member 152 and the housing 108. When the L-brace 136 is compressed, the intermediate portion 145 of the L-brace 136 is bent or lifted upward (in direction D1), the actuation component 150 lifting the connector 130 and membrane 126 upward and moving the pump mechanism 102 to the contracted configuration. The connector 130 imparts a force to the membrane 126, thereby expelling fluid out of the fluid chamber 134 through the one-way valve of the outlet 125 until the volume of the fluid chamber 134 is zero or near zero.

As the gait of the user moves to the swing phase, the prosthetic foot 104 expands and moves out of compression. The pump mechanism 102 may then return to the expanded configuration, with the top surface 131 of the membrane 126 separating from the top 122 of the interior cavity 116, increasing the volume of the fluid chamber 134. The increase in volume of the fluid chamber 134 creates a vacuum, pulling fluid from the cavity of the prosthetic socket and maintaining the hypobaric pressure chamber between the residual limb and/or liner and the prosthetic socket.

The membrane 126 may be shaped to include a bias, such that when expanding or compressive forces are not transmitted from the prosthetic foot 104 to the L-brace 136, the membrane 126 may return to an expanded configuration of FIG. 8B. Thus, the membrane 126 may be deformed during mid-stance and toe-off to move the membrane 126 against the top 122 of the interior cavity 116 in the compressed configuration, after which the membrane 126 pushes against the top 122 of the interior cavity 116 to return to the expanded configuration during swing phase.

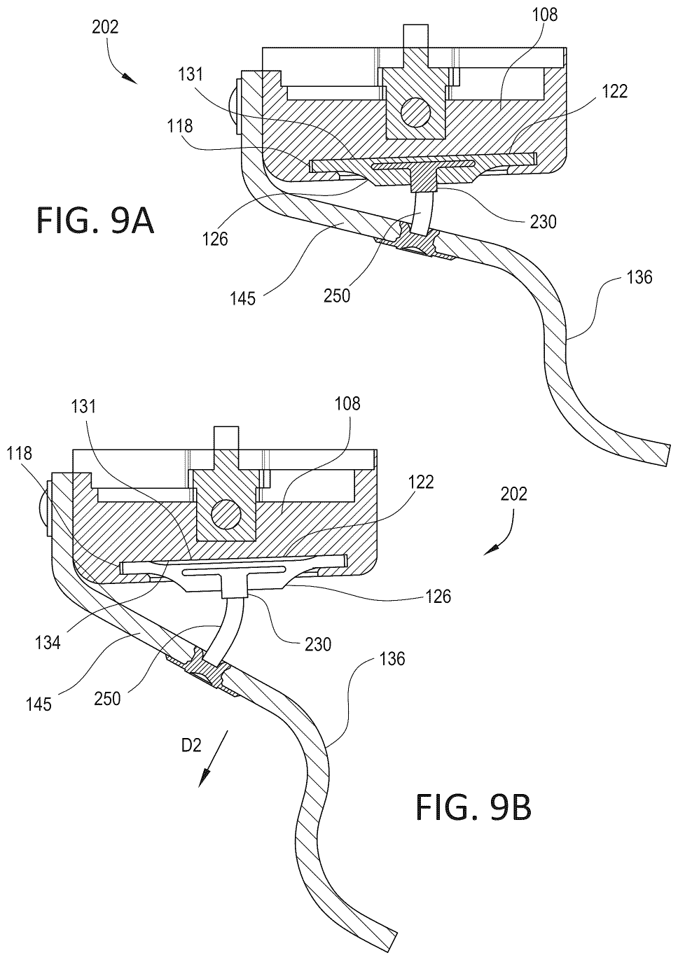

FIGS. 9A-9B show another embodiment of the pump mechanism 202, wherein an actuation component 250 attached to the L-brace 136 is also attached to the connector 230 of the membrane 126. The actuation component 250 may comprise elastic properties, such that the actuation component 250 may elongate upon expansion of the prosthetic foot 104 so as not to pull the membrane 126 out of the interior cavity 116.

FIG. 9A illustrates the pump mechanism 202 in the contracted configuration in which the volume of the fluid chamber 134 is zero or near zero. The contracted configuration of FIG. 9A may be the original configuration exhibited by the pump mechanism 202 during the swing phase. Upon heel strike, the prosthetic foot 104 moves into expansion, a distance between the proximal end 142 and distal end 144 of the prosthetic foot 104 increasing, such that the L-brace 136 extends and/or straightens because of the radius of curvature of the first curve 146 and/or the second curve 148 increasing, as seen in FIG. 9B. As the L-brace 136 extends, the intermediate portion 145 lowers (in direction D2) and the actuation component 250 attached to the L-brace 136 pulls the membrane 126 distally. In this manner, the membrane 126 is deformed, pulling the top surface 131 of the membrane 126 away from the top 122 of the interior cavity 116, increasing the volume of the fluid chamber 134. The increase in the volume of the fluid chamber 134 creates a resulting vacuum, which thereby draws fluid from the cavity of a prosthetic socket through the one-way valve 160 of the inlet 123.

As the gait of a user moves from heel strike to mid-stance and toe-off, the prosthetic foot 104 transitions from expansion to compression. The forces compressing the prosthetic foot 104 are transmitted to compress the L-brace 136, such that the intermediate portion 145 is raised with the actuation component 250 pushing against the membrane 126 to reduce the fluid chamber 134 to zero or near-zero and expelling fluid through the outlet 125 of the housing 108.

The membrane 126 can be elastomeric and can use at least in part its material properties to return to its original position naturally or elastically at the top 122 of the interior cavity 116. The membrane 126 may have any desired shape but is shown having a generally circular or elliptical shape. The membrane 126 can be attached at or near its center point to the L-brace 136. In contrast, the outer radial edge portion 128 of the membrane 126 is attached to the housing 108. When the membrane 126 is pulled away from the housing 108, a fluid chamber 134 forms in a middle area of the membrane 126 due to the deformation of the membrane 126. The pump mechanism 202 thus uses a compliant membrane 126 to create suction.

Returning to FIG. 2, the prosthetic system 100 may comprise a heel component 158. The heel component 158 may be situated between the outer foot member 154 and the heel member 156. The heel component 158 may beneficially provide additional cushioning of the prosthetic foot 104. In some embodiments, the heel component 158 may provide structural support to facilitate the expansion of the inner and outer foot members 152 and 154. The heel component 158 may contact and extend along an exterior surface 172 of the outer foot member 154 to support the outer foot member 154 during compression and/or expansion of the prosthetic foot 104.

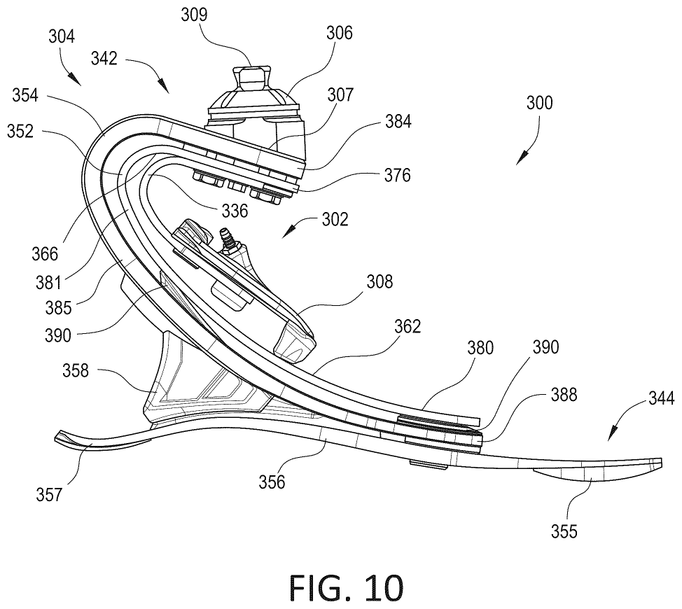

FIG. 10 illustrates another embodiment of an exemplary prosthetic system 300, including a pump mechanism 302 and a prosthetic foot 304 with dual foot blades comprising elongated plates. The prosthetic foot 304 may comprise the same or similar components as prosthetic foot 104, including inner and outer foot members 352 and 354, a heel member 356, and a heel component 358. The inner and outer foot members 352 and 354 may have intermediate portions 381 and 385 extending between first end portions 376 and 384 and second end portions 380 and 388, respectively. The intermediate portions 381 and 385 can be generally forwardly facing concave so that the inner and outer foot members 352 and 354 are generally C-shaped. The outer foot member 354 may generally surround the inner foot member 352, the outer foot member 354 being generally positioned posterior of the inner foot member 352. The first end portions 376 and 384 can be generally horizontally oriented or oriented at an oblique angle to the horizontal, and the second end portions 380 and 388 can be generally horizontally oriented. The inner and outer foot members 352 and 354 may extend generally parallel to each other have generally the same shape. Intermediate portions 381 and 385 of the inner and outer foot members 352 and 354 can have predetermined lengths to provide the prosthetic foot 304 with a desired flexibility. That is, the curvature of the outer foot member 354 can follow or be generally concentric with the curvature of the inner foot member 352.

An adaptor 306 can be coupled to the inner foot member 352 and the outer foot member 354. The inner and outer foot members 352 and 354 may be attached to a distal end 307 of the adaptor 306 through one or more fasteners. In some embodiments, the adaptor 306 can include a cavity sized and shaped to receive an attachment portion of the prosthetic foot 304 such as the first end portions 376 and 384 of the inner and outer foot members 352 and 354. The adaptor 306 can include a cavity disposed on its distal end 307. The adaptor 306 may comprise a prosthetic connection 309, such as a male pyramid connection, for connecting the prosthetic foot 304 to a prosthetic device.

The heel member 356 may extend rearwardly from a first or anterior end 355 to a free second or posterior end 357 and is disposed below at least a portion of the outer foot member 354. The heel member 356 may have a curvilinear profile along its length. The outer foot member 354 may be coupled to the heel member 356 at the second end portion 388 via fasteners. The inner foot member 352 may rest upon spacer pads 390 disposed between the inner foot member 352 and the outer foot member 354, such that the inner foot member 352 may slide relative to the outer foot member 354.

In use, the prosthetic foot 304 can expand and compress through flexion of the inner and outer foot members 352 and 354. The prosthetic foot 304 is in expansion when the first and second end portions 376 and 380 of the inner foot member 352 and the first and second end portions 384 and 388 of the outer foot member 354 are moved or flexed apart from a resting position of the prosthetic foot 304 increasing the distance between the first 376, 384 and second 380, 388 end portions of the inner and outer foot members 352 and 354. The prosthetic foot 304 is in compression when the first 376, 384 and second 380, 388 end portions of the inner and outer foot members 352 and 354 are moved or flexed toward one another from the resting position of the prosthetic foot 304, reducing the distance between the first 376, 384 and second 380, 388 end portions of the inner and outer foot members 352 and 354.

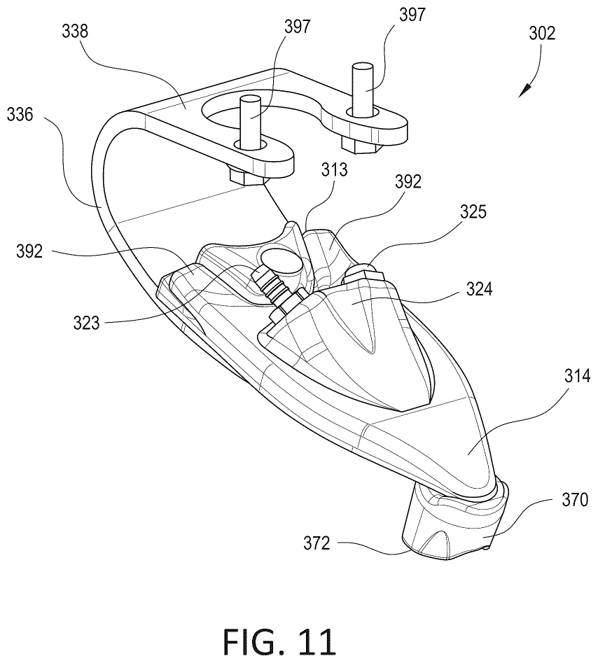

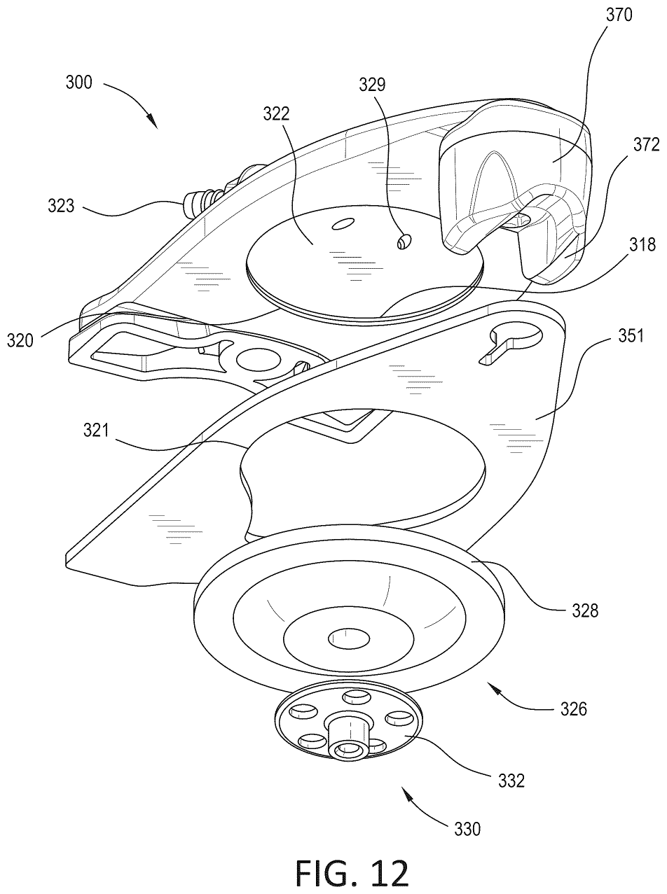

FIGS. 11 and 12 illustrate the pump mechanism 302, with FIG. 11 showing a perspective view of the pump mechanism 302. The pump mechanism 302 can be coupled to the distal end 307 of the adaptor 306 by one or more bolts 397. The pump mechanism 302 may also be coupled to the inner and outer foot members 352 and 354 at a bottom surface 366 of the inner foot member 352. The pump mechanism 302 may comprise a housing 308, a valve assembly 324, a membrane 326, a connector 330, and a C-blade 336. The C-blade 336 may be an elongate blade configured to actuate the pump mechanism 302. The C-blade 336 may comprise materials and shapes like those described in relation to L-brace 136 above. The C-blade 336 may define a curvature in a same direction as the curvature of the prosthetic foot 304 (i.e., the C-blade 336 may have a same or similar curvature as the inner and outer foot members 352 and 354 of the prosthetic foot 304). The C-blade 336 may be connected at its proximal end 338 to the proximal end 342 of the prosthetic foot 304 and extend therefrom to a distal end 340. The pump mechanism 302 may further comprise a spring blade 351 that extends along a posterior surface 310 of the housing 308 between the housing 308 and the C-blade 336. The spring blade 351 may be configured to interface between the C-blade 336 and the housing 308 to actuate the pump mechanism 302.

The housing 308 may be positioned beneath the proximal end 342 of the prosthetic foot 304. The housing 308 can have a thin elongate configuration defining an anterior surface 312 and a posterior surface 310. The housing 308 may comprise a valve assembly 324 and an interior cavity 316, similar to housing 108 described above. The valve assembly 324 may comprise one or more one-way valves, including an inlet 323 and an outlet 325. The inlet 323 may only allow fluid to enter the pump mechanism 302 and may be in fluid communication with the cavity of a socket. The outlet 325 may only allow fluid to be expelled from the pump mechanism 302, preferably to the atmosphere.

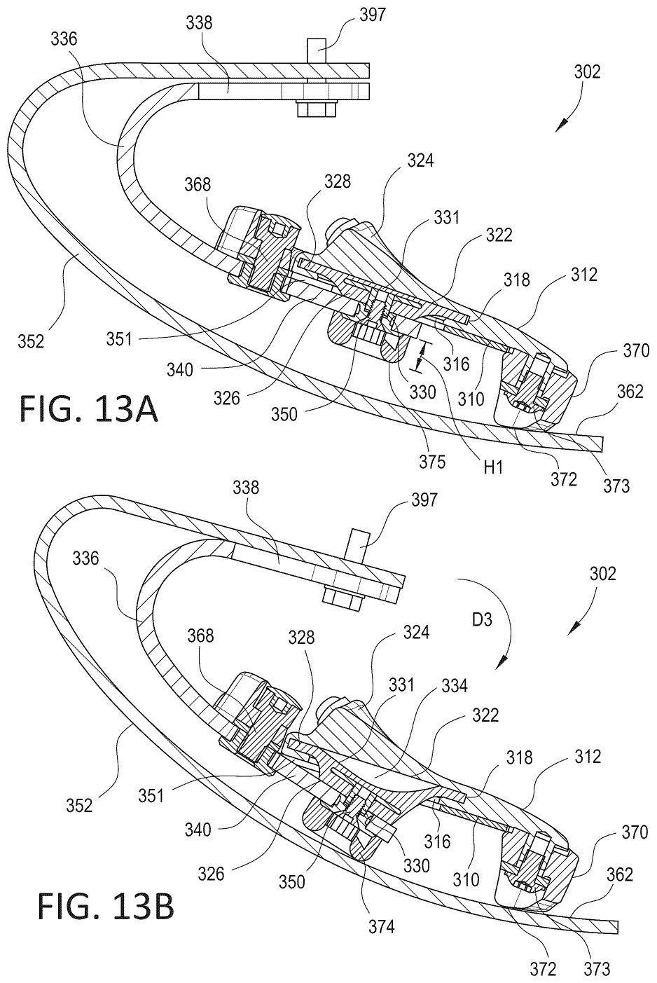

FIG. 12 illustrates an exploded view of the bottom of the housing 308 as well as the membrane 326 and the connector 330. The interior cavity 316 may extend from the posterior surface 310 of the housing 308, the interior cavity 316 defining a space for receiving a membrane 326, the interior cavity 316 and the membrane 326 together defining a fluid chamber 334 therebetween (see FIG. 13B). The interior cavity 316 may include an undercut circumferential groove 318 configured to receive an outer radial edge portion 328 of the membrane 326. The top 322 of the interior cavity 316 may define a pair of openings 329, which extend into the housing 308 to form internal passageways (not shown) to provide fluid communication between the fluid chamber 334 and the valve assembly 324. The membrane 326 may protrude from the opening 320 of the interior cavity 316 and an opening 321 formed in the spring blade 351 to connect to the distal end 340 of the C-blade 336. The membrane 326 may be connected to the C-blade 336 by a connector 330 at least partially embedded in the membrane 326. The connector 330 can have any suitable configuration and can include a radial flange 332 embedded in the membrane 326. An actuation component 350 may secure the connector 330 to the C-blade 336. The actuation component 350 may comprise a screw or other fastener.

Similar to the previously described pump mechanism 102, the pump mechanism 302 can rely on deformation of the membrane 326 to move between an original or contracted configuration in which the volume of a fluid chamber 334 defined between the top surface 331 of the membrane 326 and the top 322 of the interior cavity 316 is zero or near-zero, and an expanded configuration in which the volume of the fluid chamber 334 is increased. The housing 308 is arranged to surround the outer radial edge portion 328 of the membrane 326 and to create a seal with the membrane 326 for forming the fluid chamber 334.

The C-blade 336 may interface with the spring blade 351 and a proximal end 313 of the housing 308 and may be pivotably connected to the spring blade 351 and the housing 308 at a pivot point 368, such that the housing 308 and C-blade 336 may pivot or rotate relative to one another to actuate the pump mechanism 302. The proximal end 313 of the housing 308 may comprise one or more arms 392 to enable the housing 308 to rotate relative to the C-blade 336. The C-blade 336 can be formed of any suitable material such as a metal material, plastic, composite, and/or resin material. The C-blade 336 may also comprise carbon fiber, glass fiber, or plastic parts reinforced with carbon or glass fibers. The C-blade 336 is a plate having an elongate configuration and a distal end 340 configured to interface with the spring blade 351. A proximal end 338 of the C-blade 336 may be attached to the adaptor and/or bottom surface 366 of the inner foot member 352. The distal end 340 of the C-blade 336 may be attached to the membrane 326 via a connector 330 and an actuation component 350. The spring blade 351 is also a plate, having a generally elongate configuration, extending from the proximal end 313 of the housing 308 distally along the posterior surface 310 of the housing 308. The spring blade 351 may be clamped between the housing 308 and the C-blade 336. The distal end of the spring blade 351 may be secured to the housing 308 by a bolt 373 or other fixation device. The spring blade 351 may be configured so as to include a bias for returning the pump mechanism 302 to the contracted configuration.

The housing 308 may also define a bump component 370 extending generally downward or posterior from the posterior surface 310. The bump component 370 may be disposed, for example, at or near the distal end 314 of the housing 308. The bump component 370 can have any suitable configuration and has a lower end defining an engagement surface 372 arranged to engage a dorsal portion 362 of the prosthetic foot 304. The bump component 370 may be configured to slidably engage the dorsal portion 362 of the prosthetic foot 304. In some embodiments, the bump component 370 may be fixed to the dorsal portion 362 of the prosthetic foot 304. The engagement surface 372 may comprise a soft material or other material configured to reduce noise as the bump component 370 engages (e.g., contacts or slides along) the dorsal portion 362 of the prosthetic foot 304. For example, the engagement surface 372 of the bump component 370 may comprise a soft plastic. The bump component 370 may be formed through additive manufacturing or through injection molding processes.

FIGS. 13A-13B showing a sectional view of the pump mechanism 302 in contracted and expanded configurations, respectively. When the prosthetic foot 304 is in the resting position (as seen in FIG. 13A), the distal end 340 of the C-blade 336 may be substantially adjacent to the spring blade 351, such that the posterior surface 310 of the housing 308 lies in proximity to the distal end 340 of the C-blade 336. In this position, the fluid chamber 334 may have a zero or near-zero volume, the top surface 331 of the membrane 326 being in proximity with the top 322 of the interior cavity 316. Upon heel strike, the prosthetic foot 304 may move into expansion and the pump mechanism 302 may remain in its original configuration.

As the prosthetic foot 304 moves from heel strike through mid-stance and/or toc-off, the prosthetic foot 304 moves into compression (as seen in FIG. 13B). In compression, the proximal end 342 of the prosthetic foot 304 is pushed downward toward the distal end 344 of the prosthetic foot 304, decreasing the radius of curvature of the inner and outer foot members 352 and 354. This movement also pushes the proximal end 338 of the C-blade 336 down such that the C-blade 336 rotates forwards in a direction D3, the C-blade 336 pivoting about the pivot point 368 and the distal end 340 of the C-blade 336 rotating down towards the inner foot member 352 and away from the posterior surface 310 of the housing 308. As the distal end 340 of the C-blade 336 rotates towards the inner foot member 352 the bump component 370 pushes against the inner foot member 352 to lift the housing 308 upwards relative to the C-blade 336. As the bump component 370 lifts the housing 308, the distal end of the spring blade 351 is also lifted, the spring blade 351 bending at or near the pivot point 368 to enter a flexed state. As the distance between the housing 308 and the distal end 340 of the C-blade 336 increases, the C-blade 336 pulls the membrane 326 downward. The outer radial edge portion 328 of the membrane 326 remains in the circumferential groove 318 such that the volume of the fluid chamber 334 increases. This creates a vacuum that pulls fluid from the cavity of a socket through the inlet 323 into the fluid chamber 334.

The pump mechanism 302 may further comprise an end stop 374 disposed near the distal end 340 of the C-blade 336. The end stop 374 may comprise a soft plastic and may be formed from an additive manufacturing or injection molded process. The end stop 374 may be glued or otherwise attached near the distal end 340 of the C-blade 336. The end stop 374 functions to prevent the distal end 340 of the C-blade from separating too far from the posterior surface 310 of the housing 308 and pulling the membrane 326 out of the undercut circumferential groove 318 of the interior cavity 316. The end stop 374 may have a height H1 configured to separate the distal end 340 of the C-blade 336 from the dorsal portion 362 of the inner foot member 352 sufficient to prevent removal of the membrane 326 from the interior cavity 316.

As the prosthetic foot 304 moves from toe-off back to swing phase, the prosthetic foot 304 is moved out of compression to the original configuration, the C-blade 336 rotating backwards such that the distal end 340 of the C-blade 336 returns to the resting position. As the prosthetic foot 304 expands, the force between the bump component 370 and the dorsal portion 362 of the prosthetic foot 304 decreases. The spring blade 351 may then extend or straighten, returning to an unflexed state, so as to bring the posterior surface 310 of the housing 308 back in proximity with the distal end 340 of the C-blade 336, the top surface 331 of the membrane 326 also being brought back in proximity with the top 322 of the interior cavity 316 and thereby pushing fluid out of the fluid chamber 334 and out the outlet 325. As the prosthetic foot 304 moves from swing phase through the different stages of stance phase the pump mechanism 302 actuates and fluid is pulled from the cavity of a socket to the pump mechanism 302, thereby maintaining the hypobaric pressure chamber in the cavity of the socket.

It should be appreciated that pump mechanisms 102, 302 can be a separate add-on module to the prosthetic foot 104, 304. In addition, the pump mechanisms 102 and 302 can be adapted to fit a few different prosthetic feet, providing versatility. Because the pump mechanisms 102, 302 are not integrated into the prosthetic foot 104 or 304 failure of the pump mechanisms 102, 302 advantageously would not affect the performance of the prosthetic foot 104, 304.

It is understood that not all objects or advantages may be achieved under any embodiment of the disclosure. Those skilled in the art will recognize that the disclosed prosthetic systems may be embodied or carried out in a manner that achieves or optimizes one advantage or group of advantages as taught herein without achieving other objects or advantages as taught or suggested herein.

The skilled artisan will recognize the interchangeability of various disclosed features. Besides the variations described herein, other known equivalents for each feature can be mixed and matched by one of ordinary skill in this art to build and use prosthetic devices under the principles of the present disclosure. It will be understood by the skilled artisan that the features described herein may be adapted to other methods and types of prosthetic devices/applications.

It is intended that the present disclosure should not be limited by the disclosed embodiments described above and may be extended to other applications that may employ the features described herein.

1. A prosthetic system comprising:
a pump mechanism operatively connectable to a prosthetic foot having an adaptor, the pump mechanism including:
a housing having an interior cavity;
a one-way valve assembly connecting to the housing and in fluid communication with the interior cavity;
a membrane located at least partially within the interior cavity;
a connector attached to the membrane and protruding outside the interior cavity; and
an L-brace attached to the housing and/or the prosthetic foot and configured to cooperate with the prosthetic foot to actuate the membrane;
wherein the housing is positioned at or near a proximal end of the prosthetic foot below the adaptor.

2. The prosthetic system of claim 1, wherein relative movement of the L-brace against the prosthetic foot pulls the membrane by the connector to draw fluid through the one-way valve assembly.

3. The prosthetic system of claim 1, wherein the L-brace comprises a first curve with a radius of curvature opening towards the prosthetic foot.

4. The prosthetic system of claim 3, wherein the radius of curvature of the first curve is in a direction opposite a radius of curvature of the prosthetic foot.

5. The prosthetic system of claim 3, wherein the L-brace comprises a second curve distal of the first curve, the second curve having a radius of curvature in a direction opposite that of the first curve.

6. The prosthetic system of claim 1, wherein a proximal end of the L-brace is attached to a posterior portion of the housing.

7. The prosthetic system of claim 1, wherein a distal end of the L-brace slidably contacts a dorsal portion of the prosthetic foot.

8. The prosthetic system of claim 1, wherein a distal end of the L-brace is attached to a dorsal portion of the prosthetic foot.

9. The prosthetic system of claim 1, wherein the L-brace comprises an actuation component configured to transmit movement of the L-brace to the membrane.

10. The prosthetic system of claim 9, wherein the actuation component is attached to the connector.

11. The prosthetic system of claim 1, wherein the interior cavity is provided with an undercut circumferential groove between an open end of the interior cavity and a top of the interior cavity and wherein an outer radial edge of the membrane is situated in the undercut circumferential groove such that a seal is formed between the membrane and the housing.

12. The prosthetic system of claim 1, wherein the membrane is moveable between a contracted configuration in which volume of a fluid chamber defined between the membrane and a top of the interior cavity is zero or near-zero, and an expanded configuration in which the volume of the fluid chamber is increased.

13. The prosthetic system of claim 12, wherein when the prosthetic foot is in a resting position, the membrane is in the contracted configuration, and whereupon heel strike, the prosthetic foot moves into expansion, which in turn moves the membrane to an expanded configuration by the L-brace, which further includes a third bend having a radius of curvature arranged to increase upon heel strike.

14. The prosthetic system of claim 13, wherein the increase of the radius of curvature or straightening of the L-brace is arranged to pull the membrane away from the housing to deform the membrane between the L-brace and the housing, thereby increasing the volume of the fluid chamber and creating a vacuum in the prosthetic system by pulling fluid into the fluid chamber through the one-way valve assembly.

15. The prosthetic system of claim 14, wherein the pump mechanism is arranged so that as the prosthetic foot moves from heel strike back toward a resting position, the L-brace transitions to a compressed state, moving the membrane back toward the contracted configuration and decreasing the volume of the fluid chamber to a zero or near-zero volume, whereupon during a return of the membrane toward the housing, the pump mechanism expels fluid from the fluid chamber out of a one-way valve assembly.

16. The prosthetic system of claim 12, wherein when the prosthetic foot is in a resting position of the membrane is in the expanded configuration, and the L-brace further includes a third bend having a radius of curvature arranged to decrease upon mid-stance and/or toe-off.

17. The prosthetic system of claim 16, wherein the decrease of the radius of curvature of the third bend during mid-stance and/or toe-off is arranged to compress the L-brace and push the membrane toward the housing, thereby decreasing the volume of the fluid chamber by expelling fluid from the fluid chamber out of the one-way valve assembly.

18. The prosthetic system of claim 17, wherein the pump mechanism is arranged so that as the prosthetic foot moves from toe-off back toward a resting position, the L-brace extends, moving the membrane back toward the expanded configuration and increasing the volume of the fluid chamber to a volume greater than zero, whereupon during a return of the membrane to the expanded configuration the membrane creates a vacuum in the prosthetic system by pulling fluid into the fluid chamber through the one-way valve assembly.

19. A pump mechanism for connecting to a prosthetic foot, the pump mechanism comprising:
a housing having an interior cavity disposed on a distal surface of the housing;
a one-way valve assembly connecting to the housing and in fluid communication with the interior cavity;
a membrane located at least partially within the interior cavity; and
a connector attached to the membrane and protruding outside the interior cavity;
wherein the housing is positioned at or near a proximal end of the prosthetic foot on a bottom surface of the prosthetic foot.

20. A pump mechanism for connecting to a prosthetic foot, the pump mechanism comprising:
a housing having an interior cavity disposed on a posterior portion of the housing;
a one-way valve assembly connecting to the housing and in fluid communication with the interior cavity;
a membrane located at least partially within the interior cavity;
a connector attached to the membrane and protruding outside the interior cavity; and
a C-blade pivotably attached to the housing and configured to attach to a proximal end of the prosthetic foot.
